(19) United States
(12) Patent Application Publication
(10) Pub. No.: US 2026/0349283 A1
(43) Pub. Date: Nov. 5, 2026

(54) METHOD AND SYSTEM FOR DETERMINING FITTING PARAMETERS FOR FITTING AN OPHTHALMIC LENS IN A RIM OF A SPECTACLE FRAME FOR AN INDIVIDUAL

(71) Applicant: Essilor International, Charenton-Le-Pont (FR)

(72) Inventors: Dinesh AHANGAMA, Charenton-Le-Pont (FR); Aurélie LE CAIN, Charenton-Le-Pont (FR); Gildas MARIN, Paris (FR); Guillaume TATUR, Creteil (FR)

(73) Assignee: Essilor International, Charenton-Le-Pont (FR)

(21) Appl. No.: 19/487,310

(22) PCT Filed: Jul. 10, 2024
(86) PCT No.: PCT/EP2024/069477

§ 371 Date: Nov. 24, 2025

(30) Foreign Application Priority Data
Jul. 11, 2023 (EP) 23306181.1

(51) Int. Cl. G02C 13/00; A61B 5/11; A61B 5/16; G02B 27/00; G02B 27/01; G02C 7/02

(52) U.S. Cl. CPC G02C 13/005; A61B 5/1114; A61B 5/1127; A61B 5/163; G02B 27/0093; G02B 27/0172; G02C 7/025; G02C 7/027; G02B 2027/0138

(57) ABSTRACT

A method and system for determining fitting parameters for fitting an ophthalmic lens in a rim of a spectacle frame for an individual. Frame 3D data including points representative of said rim are first determined in a predetermined 3D reference system attached to the head of said individual. The individual is then asked to wear a head-mounted display device configured to display virtual images towards their eyes. Virtual images are displayed by said head-mounted device, and the individual is asked to adapt his posture to said at least one simulated real-life environment while an eye tracker tracks eye motions. Fitting parameters can then be determined by calculating a position of a fitting point of an ophthalmic lens relative to said spectacle frame, said fitting point corresponding to an intersection of said main gaze direction through at least an eye rotation center of said individual and said frame 3D data.

### FIELD OF THE DISCLOSURE

The present disclosure relates to a method and system for determining fitting parameters for fitting an ophthalmic lens in a rim of a spectacle frame for an individual.

### BACKGROUND OF THE DISCLOSURE

Manufacturing an ophthalmic lens adapted to an individual and a frame in order to provide the individual with appropriate ophthalmic equipment requires the determination of fitting parameters of the lens in the chosen frame.

The fitting parameters are relative to the positioning of the lens relative to the frame chosen by the wearer in order to ensure that the optical features of the lens adequately correct the visual defects of the wearer.

The positioning of the lens depends both on the geometry of the frame and the morphology of the head of the individual.

In order to ensure a correct placement of the lens in front of the eye of the wearer, both the geometrical features of the chosen frame and the morphological features of the individual have to be taken into account.

This is why the fitting parameters are preferably relative to a given frame and a given individual. They are determined on the basis of measurements performed on the individual wearing the chosen frame.

In order to fit a lens in a frame, it is desirable to determine the position of a specific point of the lens relative to the frame, and at least one angle of orientation of the lens relative to the frame about an axis globally perpendicular to a mean plane of the rim of the frame or to a mean plane of the lens when mounted inside the frame. In practice, the specific point of the lens used to fit the lens is for example the fitting point P shown in FIG. 1.

The fitting point P is a point of the front surface of the lens L stipulated by the manufacturer as a reference point for positioning the lens in front of the eye of the individual. The fitting point position is determined by measuring the vertical and horizontal fitting distances of the fitting point from the boxed center BC of the lens shape (see FIG. 1).

The boxed center BC is defined as the center of the boxing system BS associated with the lens L. As shown on FIG. 1, this boxing system BS is a referential of the lens L, based on the rectangle formed by the horizontal and vertical tangents to the outer most edges of the shape of the lens. The sides of the box are aligned with the horizontal and vertical directions of the lens when placed in a frame on the head of an individual.

The shape of the lens is the outline of the lens in a mean plane of the lens.

In other words, the lens shape is the shape of the edge of the lens projected in a plane when it has been cut to fit inside the frame: the edge of the lens then corresponds precisely to the inner periphery of the circle of the frame, when the frame comprises circles.

When the frame does not comprise circles, the lens shape is that of the lens blank associated to the frame.

FIG. 1 shows the main elements of this boxing system BS for the left lens L whose shape is shown on FIG. 1. Corresponding elements may be defined for the right lens L1.

The standard measurements of fitting parameters use the sides of the boxes as reference lines.

The boxes center BC is the center of the rectangle defined above. The vertical centerline VC is the straight line going through the boxed center and parallel to the side of the rectangle.

The fitting point P position is determined by the fitting point height FH and fitting point horizontal distance to the boxed center D2 or to a vertical side of the box system D1 of the lens L or the horizontal distance D3 to a vertical symmetry axis X of the boxing system BS. The vertical symmetry axis X of the boxing system BS is defined as the axis of symmetry between the rectangles of the boxing system for the right and left lens when the lenses are placed relative to each other as they would be in the chosen frame.

The fitting point height FH is the vertical distance of the fitting point above the horizontal tangent to the lens shape at its lowest point. The fitting parameters may comprise, among others, the position of the fitting point P, the fitting point height FH, the interpupillary distance and the right and left monocular pupillary distances of the individual.

For an accurate fitting of the lens in the frame for the individual, the fitting point height FH must match the vertical distance between the pupil of the individual and the bottom edge of the lens in the frame when worn, and the distance between the fitting point and the vertical symmetry axis X must match the monocular pupillary distance of the individual.

In order to be accurate, the measurement of the fitting parameters such as the fitting height requires the individual to be in his natural posture. The natural posture is assumed by the individual when he looks straight ahead to the horizon without any visual or postural constraint. This natural posture is also called orthostatic posture and corresponds to the position in which the individual achieves minimal efforts.

In this natural posture, the Frankfurt plane of the head is horizontal. The Frankfurt plane is defined as the plane passing through the inferior margin of an orbit (for example, the point called the left orbital) and the upper margin of each ear canal or external auditory meatus, a point called the porion.

Nowadays, all measurements for fitting including the validation step (including the validation at the EG delivery), are done in non-natural environments and posture (typically in an optician shop in front of a measurement system such as Visioffice®, Eyeruler® or M'eye fit®), resulting in non-adapted equipment.

Wearers complain about non-adaptation that are due for 40% to problems in the fitting (2nd factor after prescription errors of non-adaptation). Those problems may be due to measurement conditions that are far from the natural ecological environment and posture. Indeed, the current test conditions evaluate the fitting parameters in specific conditions. However, it is known that the measurement conditions favor a non-natural posture (standing of attention!).

Therefore, there is a need to find solutions to perform the measurements in more natural conditions.

### SUMMARY OF THE DISCLOSURE

To that end, the disclosure provides a method for determining fitting parameters for fitting an ophthalmic lens in a rim of a spectacle frame for an individual, said method comprising:
determining frame 3D data in a predetermined 3D reference system attached to the head of said individual, said frame 3D data including points representative of said rim;
having said individual wearing a head-mounted display device configured to display virtual images towards the eyes of the individual;
displaying virtual images representatives of at least one simulated real-life environment using said head-mounted display device, and having said individual to adapt his posture to said at least one simulated real-life environment;
while said individual adapts his posture:
   tracking eye motions of said individual using an eye tracker device embedded in said head-mounted display device;
   determining a main gaze direction in said predetermined 3D reference system based on the tracked eye motions; and
determining said fitting parameters by calculating a position of a fitting point of an ophthalmic lens relative to said spectacle frame, said fitting point corresponding to an intersection of said main gaze direction going through at least an eye rotation center of said individual and said frame 3D data.

In particular embodiments, determining frame 3D data may comprise capturing images of said individual not wearing said spectacle frame, and acquiring said frame 3D data from a database. Alternatively, in other embodiments, determining frame 3D data may comprise capturing images of said individual wearing said spectacle frame.

In both cases, the method further comprises extracting the origin of the 3D reference system by processing the captured images.

In particular embodiments, determining frame 3D data may comprises tracking the motions of the head of said individual using a tracking device.

An origin of the 3D reference system can be e.g., an eye rotation center, or a cyclopean eye, or a facial feature of said individual.

In some embodiments, determining a main gaze direction comprises measuring a plurality of gaze directions for a plurality of tasks, and said main gaze direction is an average of the plurality of measured gaze directions.

In particular embodiments, determining a main gaze direction in the predetermined 3D reference system may comprise:
determining first said main gaze direction with respect to a 3D coordinate system attached to said head-mounted display device based on the tracked eye motions;
tracking the head of said individual and said head-mounted display device in said predetermined 3D system in order to determine a relative position of said 3D coordinate system with respect to said predetermined 3D reference system; and
converting said main gaze direction into said predetermined 3D reference system based on the determined relative position.

In some embodiments, the method may further comprise a calibration step consisting in:
measuring a position of the head of the individual not wearing said head-mounted display device;
measuring a position of the head of the individual when wearing said head-mounted display device;
calculating a bias for said head-mounted display device based on the positions of the head measured with and without said head-mounted display device.

In particular embodiments in this case, said main gaze direction is converted into said predetermined 3D reference system based on the determined relative position and the calculated bias.

In particular embodiments: the method may further comprise applying a simulated correction using said head-mounted display device.

The present disclosure also provides a system for determining fitting parameters for fitting an ophthalmic lens in a rim of a spectacle frame for an individual, said system comprising:
means for determining frame 3D data in a predetermined 3D reference system attached to the head of said individual, said frame 3D data including points representative of said rim;
a head-mounted display device configured to display virtual images towards the eyes of the individual wearing said head-mounted device, wherein said virtual images are representatives of at least one simulated real-life environment;
an eye tracker device embedded in said head-mounted display device, said eye tracker device being configured to track eye motions of said individual while said individual adapts his posture to said at least one simulated real-life environment:
processing means configured for
   determining a main gaze direction in said predetermined 3D reference system based on the tracked eye motions; and
   determining said fitting parameters by calculating a position of a fitting point of an ophthalmic lens relative to said spectacle frame, said fitting point corresponding to an intersection of said main gaze direction going through at least an eye rotation center of said individual and said frame 3D data.

In some embodiments, said means for determining frame 3D data comprise a camera for capturing images. In other embodiments, said means for determining frame 3D data comprise a tracking device configured to track the motions of the head of said individual.

In some embodiments, means for determining frame 3D data comprise a database.

In some embodiments, the system may further comprise:
first means for measuring a position of the head of the individual not wearing said head-mounted display device;
second means for measuring a position of the head of the individual when wearing said head-mounted display device;
processing means for calculating a bias for said head-mounted display device based on the positions of the head measured with and without said head-mounted display device.

For these embodiments, said first means may comprise an object configured to be worn on the head of said individual by said individual, said object supporting artificial markers. Said second means may comprise a landmark tracker integrated in said head-mounted display device, said landmark tracker being configured to track physiological landmarks of said individual.

### BRIEF DESCRIPTION OF THE DRAWINGS

For a more complete understanding of the description provided herein and the advantages thereof, reference is now made to the brief descriptions below, taken in connection with the accompanying drawings and detailed description, wherein like reference numerals represent like parts.

FIG. 1, already described above, is a schematic view of a lens with its boxing system and some fitting parameters;

FIG. 2 is a flow chart illustrating different steps of a method for determining fitting parameters, according to one possible embodiment of the invention;

FIG. 3 shows three views illustrating schematically the principles of the method according to the invention.

FIG. 4 gives views showing possible equipments for tracking the head of an individual in a system according to one embodiment.

### DETAILED DESCRIPTION OF THE DISCLOSURE

In the description which follows, the drawing figures are not necessarily to scale and certain features may be shown in generalized or schematic form in the interest of clarity and conciseness or for informational purposes. In addition, although making and using various embodiments are discussed in detail below, it should be appreciated that as described herein are provided many inventive concepts that may embodied in a wide variety of contexts. Embodiments discussed herein are merely representative and do not limit the scope of the disclosure. It will also be obvious to one skilled in the art that all the technical features that are defined relative to a process can be transposed, individually or in combination, to a device and conversely, all the technical features relative to a device can be transposed, individually or in combination, to a process and the technical features of the different embodiments may be exchanged or combined with the features of other embodiments.

The terms “comprise” (and any grammatical variation thereof, such as “comprises” and “comprising”), “have” (and any grammatical variation thereof, such as “has” and “having”), “contain” (and any grammatical variation thereof, such as “contains” and “containing”), and “include” (and any grammatical variation thereof such as “includes” and “including”) are open-ended linking verbs. They are used to specify the presence of stated features, integers, steps or components or groups thereof, but do not preclude the presence or addition of one or more other features, integers, steps or components or groups thereof. As a result, a method, or a step in a method, that “comprises”, “has”, “contains”, or “includes” one or more steps or elements possesses those one or more steps or elements, but is not limited to possessing only those one or more steps or elements.

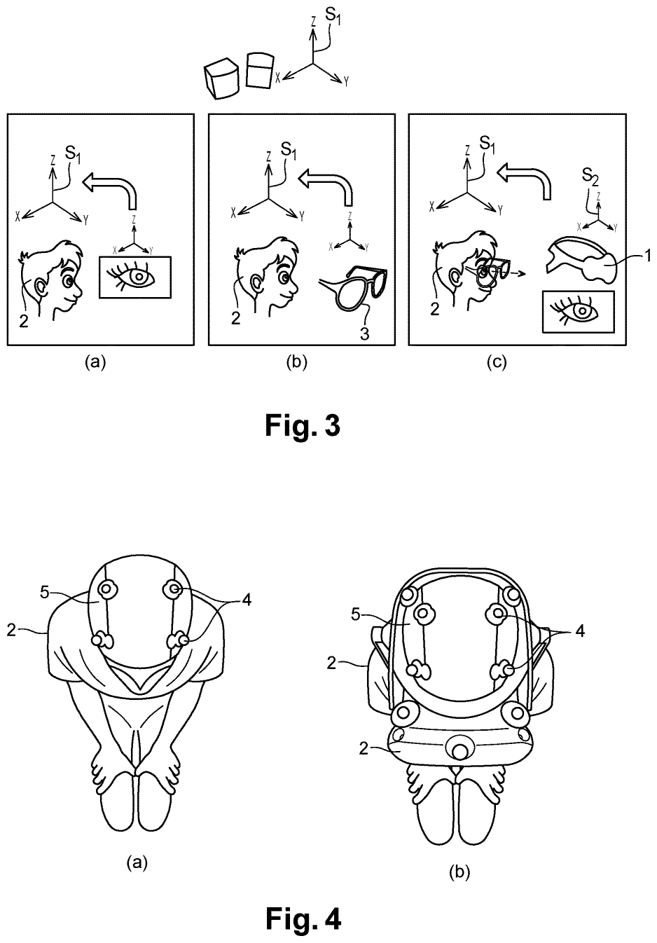

The method according to the invention is mainly based on the use of a system comprising a head-mounted display device 1 (see FIGS. 3 and 4) configured to display virtual images towards the eyes of an individual 2 wearing said head-mounted device, wherein said virtual images are representatives of at least one simulated real-life environment. The head-mounted device 1 is for instance a virtual reality or a mixed reality headset or an extended reality headset. The simulated real-life environments may be defined according to the life-style of the wearer. Various personalized environments corresponding to various situations (driving, walking . . . ) especially in FV (adapted to the primary gaze direction) can be set. It is possible to adapt the environment to the age/category of the wearer and propose to the wearer to fully personalize the environment in order to reproduce its home for example. It is possible to use mixed reality to keep some part of the real environment and to augment or replace it with virtual parts/objects. In addition, mixed reality may make the interaction with the eye care professional easier. Head-mounted display device 1 also integrates an eye tracker device configured to track eye motions of an individual wearing the head-mounted device.

The fitting measurement implies that we estimate the main gaze direction in the frame coordinate system. Here, we propose to use an external tracking system, and an eye tracker device embedded in the head-mounted device 1, to measure both the spectacle frame (in real environment) and the gaze direction (in virtual environment in a headset) in a same predetermined 3D reference system S₁ attached to the head of the individual 2.

3D reference system S₁ can be any arbitrary reference system attached to the head, or to one eye or to both eyes of the individual.

In one embodiment, the origin of the 3D reference coordinate system S₁ can be an eye rotation center.

In another embodiment, the origin of the 3D reference system S₁ can be a cyclopean eye, i.e., a theoretical eye located on the midline between the center of rotation of the left eye and the center of rotation of the right eye.

In still another embodiment, the origin of the 3D reference system S₁ can corresponds to any physiological landmarks such as a facial feature (e.g., lips contour or commissures, nose or chin tip . . . ) of the individual.

Whatever the origin chosen for the 3D reference system S₁, the system S₁ is further defined by three perpendicular axes crossing said origin. For instance, a first horizontal axis may be defined as a horizontal axis straight ahead (corresponding to an initial supposed primary eye gaze estimation being the horizontal line linking the chosen origin with a fixation target when looking straight ahead). The two other axes are thus defined as being the horizontal and vertical axes perpendicular to the first axis.

Another possibility is to use for one of the three axes the orientation defined by the axis that goes through the two eye rotation centers. The other axes are chosen the same way as the first example.

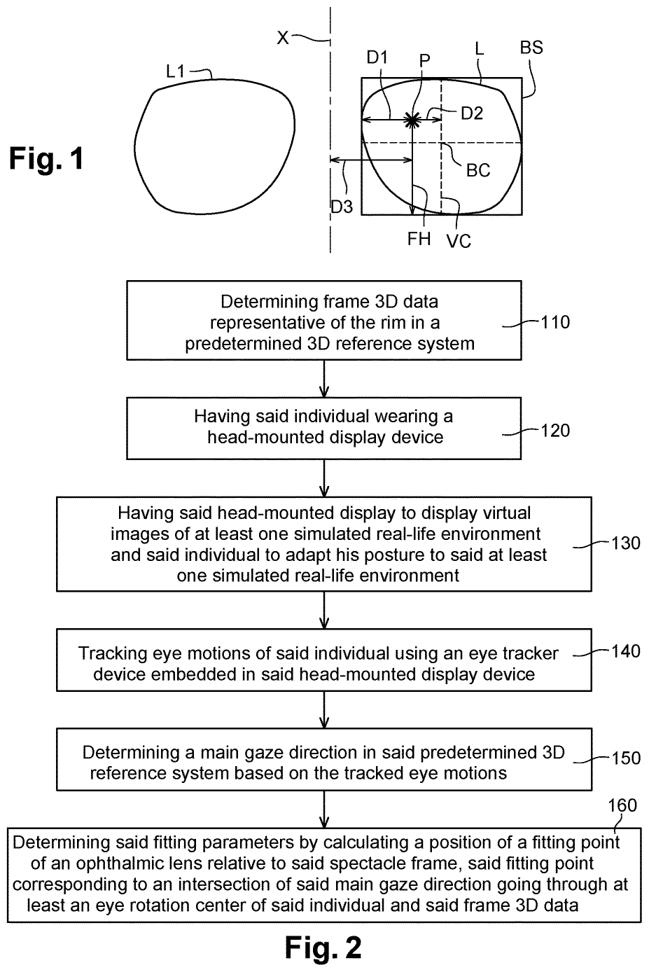

Different steps of a method 100 according to the invention for determining fitting parameters for fitting an ophthalmic lens in rim 3 of a spectacle frame for individual 2, are now detailed below, with respect to FIGS. 2 and 3. In the following, it is assumed that individual 2 has first chosen in the optical shop a specific model of spectacle frame.

For the purpose of the invention, “rim” means the perimeter of the spectacle lens where a traditional physical rim would go, regardless of whether there is actually a physical rim.

At step 110, frame 3D data including a set of frame points representative of rim 3 are determined in the 3D reference system S₁ attached to the head of individual 2.

In some embodiments, the frame points of the set are part of the real contour of rim 3. In other possible embodiments, the frame points of the set belong to the perimeter of the rectangle which defines the boxing system BS of the ophthalmic lens to be fitted in rim 3.

For the determination of the set of frame points, the individual 2 may be asked to wear the chosen spectacle frame. In this case, the set of frame points can be calculated by processing images of individual 2 wearing the spectacle frame, captured by an external object positioning system using an external camera and computer vision algorithm.

Alternatively, the individual 2 does not have to wear the chosen spectacle frame. In this case, the set of frame points can still be calculated by processing images of individual 2 not wearing the spectacle frame, as captured by the external positioning system, with a virtual fitting based on physiological landmarks of the individual or a 3D model of the head, of a virtual spectacle frame acquired from a database.

On both cases, the origin of the reference system S₁ is also extracted by extracting the captured images.

In some embodiments, the set of frame points can be determined by tracking the motions of the head of individual 2 using a tracking device.

At the end of step 110, the system has extracted the 3D coordinates of the set of frame points in the reference system S₁ attached to the head.

Next, the individual 2 is asked at step 120 to wear head-mounted display device 1. As stated above, the head-mounted display device 1 is configured to display virtual images towards the eyes of individual 2 wearing said head-mounted device, wherein said virtual images are representatives of one or several simulated real-life environments.

At step 130, the virtual images representative of each chosen simulated real-life environment are displayed, and the individual 2 is asked to adapt his posture (distance, seating posture, head/trunk/arm/hand position and orientation . . . ) to the proposed simulated real-life environment.

While said individual adapts his posture for a given simulated real-life environment, the following steps 140, 150 and 160 are performed:

The first step 140 consists in tracking eye motions of the individual 2 using an eye tracker device embedded in the head-mounted display device 1.

A main gaze direction is then determined in the 3D reference system S₁, at step 150, based on the tracked eye motions. More specifically a main gaze direction can be determined in the 3D reference system S₁ by:
determining first said main gaze direction with respect to a 3D coordinate system S₂ attached to said head-mounted display device 1 based on the tracked eye motions (see FIG. 3(c));
tracking the head of said individual and said head-mounted display device 1 in the 3D reference system S₁ in order to determine a relative position of said 3D coordinate system S₂ with respect to said 3D reference system S₁; and
converting said main gaze direction into 3D reference system S₁ based on at least the determined relative position.

In some embodiments, a plurality of gaze directions for a plurality of tasks are measured. For instance, far vision tasks may be alternated with tasks at different distances. The main gaze direction is then calculated as being an average of the plurality of measured gaze directions.

Then, at step 160, the position of the fitting point P of the ophthalmic lens relative to the chosen spectacle frame can be calculated, by finding the intersection (using ray tracing) of the main gaze direction going through at least an eye rotation center of said individual and the frame 3D data. Once the position of the fitting point P is known, the other fitting parameters can be determined, as explained in the introduction with respect to FIG. 1.

One can note that in addition to the standard protocol for fitting measurement, we can also measure the heatmap of the eye gaze or other postural and behavioral parameters. These additional measurements could be used to derive personalization parameters to adjust the design accordingly.

In some embodiments, in addition to steps 110 to 160 shown in FIG. 2, the method according to the invention may comprise a calibration step for taking into account the weight of the headset 1 on the head posture and compensate for the bias in headset measurements. This calibration step (not represented in FIG. 2) consists mainly in:
measuring a position of the head of the individual 2 not wearing the head-mounted display device 1;
measuring a position of the head of the individual 2 when wearing said head-mounted display device 1; and
calculating, via processing means, a bias for said head-mounted display device 1 based on the positions of the head measured with and without said head-mounted display device 1.

As has just been mentioned, the calibration step is performed to take into account the weight of the headset 1 on the head posture and to compensate for the bias in headset measurements. Thus, the calculated bias may be taken into consideration into one or more steps of the method according to the invention which are subsequent to the calibration step. For instance, in some embodiments, at step 150, the main gaze direction is converted into 3D reference system S₁ based on the determined relative position and the calculated bias.

To determine headset position with respect to the head, the head needs to be tracked at the same time of the head-mounted display device 1, either by the external object positioning system or by the head-mounted display device 1 itself.

In practice, a system suitable for implementing the method according to the invention needs first means for measuring a position of the head of the individual not wearing the head-mounted display device 1, and second means for measuring a position of the head of the individual when wearing said head-mounted display device 1.

To this end, according to some embodiments, artificial markers or physiological landmarks can be attached to the head and tracked with an external tracking device. The external tracking device can be known devices from Vicon or ART, adapted to use markers/landmarks in visible or infra-red range, or can be any other like magnetic tracking device, e.g., from Polhemus, to avoid the problem of visibility, or any combination of these devices. FIG. 4 shows a possible embodiment of the system in which artificial markers 4 are supported by an object 5 conceived to be worn on the individual's head. Object 5 can be a soft clothing, e.g., a cap as illustrated in view (a) of FIG. 4, or a rigid object as illustrated in view (b) of FIG. 4.

In another possible embodiment, markers/landmarks and head-mounted device can be tracked externally, with an artificial intelligence algorithm trained to identify a combination of the head and the head-mounted device).

In still another possible embodiment, physiological landmarks such as eye, eye corners, commissures and/or eyebrow can be tracked through a landmark tracker integrated in the head-mounted device.

The system for implementing the method illustrated in FIG. 2 can also be used to validate/demonstrate the fitting result on a given PAL design. Far Vision prescription is applied to the optics of the headset at step 130. A progressive correction may be simulated by the headset above the prescription correction. Individual 2 is asked to wear this headset in order to be immersed in realistic environments reproducing various ecological situations (lighting conditions, night driving visual conditions, freedom of motion, geometric layout of the scene, . . . ) to validate the correct fitting in those situations. Simulation may exaggerate the optical defects, the objective is not to perfectly render the real vision through the lens but to highlight the position of the main zone of the design (Far Vision, Intermediate Vision, Near Vision) to allow the individual to validate the correct positioning of the lens.

1. A method for determining fitting parameters for fitting an ophthalmic lens in a rim of a spectacle frame for an individual, said method comprising:
determining frame 3D data in a predetermined 3D reference system attached to a head of the individual, the frame 3D data including points representative of the rim;
having the individual wearing a head-mounted display device configured to display virtual images towards eyes of the individual;
displaying virtual images representatives of at least one simulated real-life environment using the head-mounted display device, and having the individual adapt a posture to the at least one simulated real-life environment;
while the individual adapts the posture:
tracking eye motions of the individual using an eye tracker device embedded in the head-mounted display device;
determining a main gaze direction in the predetermined 3D reference system based on the tracked eye motions; and
determining said fitting parameters by calculating a position of a fitting point of an ophthalmic lens relative to the spectacle frame, the fitting point corresponding to an intersection of the main gaze direction going through at least an eye rotation center of the individual and the frame 3D data.

2. A method according to claim 1, wherein determining frame 3D data comprises capturing images of the individual not wearing the spectacle frame, and acquiring the frame 3D data from a database.

3. A method according to claim 1, wherein determining frame 3D data comprises capturing images of the individual wearing the spectacle frame.

4. A method according to claim 2, further comprising extracting an origin of the 3D reference system by processing the captured images.

5. A method according to claim 1, wherein determining frame 3D data comprises tracking a motions of the head of the individual using a tracking device.

6. A method according to claim 1, wherein an origin of the 3D reference system is an eye rotation center, or a cyclopean eye, or a facial feature of the individual.

7. A method according to claim 1, wherein determining a main gaze direction comprises measuring a plurality of gaze directions for a plurality of tasks, wherein the main gaze direction is an average of the plurality of measured gaze directions.

8. A method according to claim 1, wherein determining a main gaze direction in the predetermined 3D reference system comprises:
determining first the main gaze direction with respect to a 3D coordinate system attached to the head-mounted display device based on the tracked eye motions;
tracking the head of the individual and the head-mounted display device in the predetermined 3D reference system in order to determine a relative position of the 3D coordinate system with respect to the predetermined 3D reference system; and
converting the main gaze direction into the predetermined 3D reference system based on the determined relative position.

9. A method according to claim 1, further comprising a calibration step consisting in:
measuring a position of the head of the individual not wearing the head-mounted display device;
measuring a position of the head of the individual when wearing the head-mounted display device;
calculating a bias for the head-mounted display device based on the positions of the head measured with and without the head-mounted display device.

10. A method according to claim 1, further comprising applying a simulated correction using the head-mounted display device.

11. A system for determining fitting parameters for fitting an ophthalmic lens in a rim of a spectacle frame for an individual, the system comprising:
means for determining frame 3D data in a predetermined 3D reference system attached to a head of the individual, the frame 3D data including points representative of the rim;
a head-mounted display device configured to display virtual images towards the eyes of the individual wearing the head-mounted device, wherein the virtual images are representatives of at least one simulated real-life environment;
an eye tracker device embedded in the head-mounted display device, the eye tracker device being configured to track eye motions of the individual while the individual adapts a posture to the at least one simulated real-life environment:
processing means configured for
determining a main gaze direction in the predetermined 3D reference system based on the tracked eye motions; and
determining the fitting parameters by calculating a position of a fitting point of an ophthalmic lens relative to the spectacle frame, the fitting point corresponding to an intersection of the main gaze direction going through at least an eye rotation center of the individual and the frame 3D data.

12. A system according to claim 11, wherein the means for determining frame 3D data comprise a camera for capturing images or a tracking device configured to track a motions of the head of the individual.

13. A system according to claim 12, wherein the means for determining frame 3D data comprise a database.

14. A system according to claim 11, further comprising:
first means for measuring a position of the head of the individual not wearing the head-mounted display device;
second means for measuring a position of the head of the individual when wearing the head-mounted display device;
processing means for calculating a bias for the head-mounted display device based on the positions of the head measured with and without the head-mounted display device.

15. A system according to claim 14, wherein the first means comprise an object configured to be worn on the head of the individual, the object supporting artificial markers.

16. A system according to claim 14, wherein the second means comprise a landmark tracker integrated in the head-mounted display device, the landmark tracker being configured to track physiological landmarks of the individual.
